(19) United States
(12) Patent Application Publication
(10) Pub. No.: US 2015/0328260 A1
(43) Pub. Date: Nov. 19, 2015

(54) BLOOD CELL PREPARATIONS AND RELATED METHODS (GEN 8)

(71) Applicant: Robert CHOW, Irvine, CA (US)

(72) Inventor: Robert Chow, Irvine, CA (US)

(21) Appl. No.: 14/653,201

(22) PCT Filed: Nov. 18, 2013
(86) PCT No.: PCT/US2013/070507

§ 371 Date: Jun. 17, 2015

Related U.S. Application Data
(60) Provisional application No. 61/738,966, filed on Dec. 18, 2012.

(51) Int. Cl. A61K 35/18; A61K 35/15

(52) U.S. Cl. CPC A61K 35/18; A61K 35/15; A61K 2035/124

(57) ABSTRACT

The disclosure provides preparations of cells that are enriched in white blood cells, methods for separating cells into different fractions, and methods for administering the different cell fractions into a recipient subject.

### CROSS-REFERENCE TO RELATED APPLICATIONS

This application claims the full Paris Convention benefit of and priority to, U.S. provisional application Ser. No. 61/738,966 m filed Dec. 18, 2013, the contents of which are incorporated by this reference as if fully set forth herein in their entirety.

### FIELD OF THE DISCLOSURE

The present disclosure relates to placental neonatal blood, also known as umbilical cord blood, placental neonatal blood, fetal blood, or placental blood. The disclosure also relates to fractions of placental neonatal blood that are enriched in white blood cells, or enriched in red blood cells, as well as in fractions that are reduced in red blood cells, reduced in white blood cells, reduced in plasma, depleted of plasma, or in fractions that are comprised mostly of plasma. The disclosure provides blood cell compositions that are prepared, for example, by centrifugation or other techniques of cell separation, cryoprotection, freezing, and thawing, and to methods for administering blood cell compositions to a subject.

### BACKGROUND OF THE DISCLOSURE

Placental neonatal blood is useful for treating a number of disorders. Placental neonatal blood cells can be cryogenically stored for future use in the same subject or recipient as the donor subject (autologous transfer). Syngeneic transfer (or transplant) is transfer (or transplant) from an identical twin. Most commonly used, is placental neonatal blood cells acquired from an allogeneic transplant donor, and then transplanted into a person different from the donor (allogenic transfer). The donor can be related (related allogenic transplantation) or to unrelated (unrelated allogenic transplantation) to the recipient. From a single umbilical cord, about 50 mL to 500 mL of blood can be acquired and used for transplantation into a recipient. For transplantation, placental neonatal blood from more than one donor can be combined, placental neonatal blood can be combined with other sources of hematopoietic stem cells, or white blood cells from a single donor can be expanded, and then transplanted into a recipient.

Where bone marrow or peripheral blood, rather than placental neonatal blood, is the source of hematopoietic stem cells and progenitor cells (which are contained in the white blood cell fraction), it is traditional to use marrow or peripheral blood from a donor who is HLA-matched. HLA-matched is preferred for at least 10-12 out of 12 HLA A/B/C/DP/DQ/DR alleles. The HLA-matched donor can be autologous, a sibling, another related donor, or an unrelated donor. However, only about 30% of patients have a sibling donor who can meet the stringent matching requirements. In absence of a matched sibling donor, the patient can rely on a network of bone marrow registries to find an HLA-matched donor (Brown et al (2008) Clin. Immunol. 127:286-297; Seggewiss et al (2010) Blood 115:3861-3868). Placental neonatal blood is an alternative source of hematopoietic stem cells and progenitors. HLA-matching is less critical with greater or equal to four matches out of six HLA A/B/DR loci. HLA-matching can be less critical, because placental neonatal blood transplants present a lesser risk for acute and chronic GVHD. Bone marrow transplants or peripheral blood stem cell transplants pose a greater risk for GVHD in frequency and severity (see, e.g., Petropoulou and Rocha (2011) Stem Cells Int. 2011:610514 (8 pages); Narimatsu (2011) Stem Cells Int. 2011:607569 (6 pages)).

Indications for placental neonatal blood transplants include, without implying any limitation, hematological cancers, genetic diseases, autoimmune disorders, and regenerative medicine, e.g., Krabbe's disease, myocardial infarction, diabetes, and stroke. Indications for the reagents of the present disclosure also include human immunodeficiency virus (HIV) infections, as demonstrated by the success with “the Berlin patient.” The Berlin patient study is notable in that it resulted in a potential functional cure for HIV (Johnston et al (2012) J. Int. AIDS Soc. 15:16 (7 pages). Hematological cancers include lymphoid neoplasms, that is, acute lymphocytic leukemia (ALL), chronic lymphocytic leukemia (CLL), and hairy cell leukemia (HCL). Hematological cancers also include myeloid neoplasms, that is, acute myeloid leukemia (AML), acute promyelocytic leukemia (APL), chronic myeloid leukemia (CML), and myelodysplastic syndromes (MDS). Placental neonatal blood contains pluripotent cells that can differentiate into all three lineages (ectoderm, mesoderm, and endoderm) in the body, including neural, cardiac, epithelial, hepatocytic, and dermal tissue (van de Ven et al (2007) Exp. Med. 35:1753-1765). The above indications are targeted by blood cell compositions of the present disclosure.

### SUMMARY OF THE DISCLOSURE

What is provided is a method for providing fractions from a whole placental neonatal blood composition, wherein the method provides a white blood cell-enriched fraction and a red blood cell (RBC)-enriched fraction, and optionally a plasma fraction, wherein the RBC-enriched fraction contains more than one white blood cell, wherein the sum of: (i) the number of white blood cells in the white blood cell-enriched fraction plus (ii) the number of white blood cells in the RBC-enriched fraction is at least 90% (or at least 50%, at least 60%, at least 70%, at least 75%, at least 80%, at least 85%, at least 95%, at least 98%, or at least about 100%) of the total number of white blood cells in the whole placental neonatal blood composition, and wherein the number of white blood cells in the sum is corrected for any samples that are withdrawn for archival or testing purposes, the method comprising: (a) processing the whole placental neonatal blood composition to provide a white blood cell-enriched fraction and a RBC-enriched fraction, using a device that is capable of separating cells into the white blood cell-enriched fraction and the RBC-enriched fraction, and optionally the plasma fraction, (b) cryogenically storing the cells of the white blood cell-enriched fraction and cryogenically storing the cells of the RBC-enriched fraction, and optionally the plasma fraction, (c) wherein the stored cells of the white blood cell-enriched fraction and the stored cells of the RBC-enriched fraction are capable of administration to one recipient, and wherein said administration is capable of transferring into the recipient at least 90% of the white blood cells, that were derived from the whole placental neonatal blood composition.

Also provided is the above method, wherein the device comprises a centrifuge or a cell fractionator. Also provided is the above method, wherein separation is effected by contacting the whole placental neonatal blood composition with a chemical composition that is capable of separating the whole placental neonatal blood composition into a white blood cell-rich fraction and a RBC-rich fraction, and optionally the plasma fraction.

Also provided is the above method, wherein separation is effected by contacting the whole placental neonatal blood composition with a chemical composition that is capable of separating the whole placental neonatal blood composition into a white blood cell-rich fraction and a RBC-rich fraction, and optionally the plasma fraction, and wherein the chemical composition comprises hydroxyethyl starch, density gradient medium, or an antibody.

Also provided is the above method, wherein the whole placental neonatal blood composition comprises an anti-coagulant. Also provided is the above method, wherein the archival or testing purposes comprises one or more of a hematology test, a blood chemistry test, and a donor identification test. What is also embraced, is the above method, wherein cells of the white blood cell-rich fraction are administered to the recipient, followed by cells of the RBC-rich fraction being separately administered to the same recipient.

Also contemplated, is the above method, wherein cells of the RBC-rich fraction are administered to the recipient, followed by the white blood cells of the white blood cell-rich fraction being separately administered to the same subject. Also provided is the above method, wherein the whole placental neonatal blood composition comprises one or more anticoagulants. Furthermore, what is provided is the above method, wherein the whole placental neonatal blood composition comprises one or more anticoagulants, and wherein the one or more anticoagulants is one or more of citrate and heparin. Also provided is the above method, wherein the cells of the white blood cell-enriched fraction are processed by washing to reduce concentration of free hemoglobin, wherein the washing occurs after thawing the cells of the white blood cell-enriched fraction and before administering the cells of the white blood cell-enriched fraction to a recipient. In another aspect, what is provided is the above method, wherein the cells of the RBC-enriched fraction are processed by washing to reduce concentration of free hemoglobin, wherein the washing occurs after thawing the cells of the RBC-enriched fraction and before administering the cells of the RBC-enriched fraction to a recipient.

Also provided is the above method, wherein one or both of the cells from the white blood cell-enriched fraction and the RBC-enriched fraction are not washed, wherein after thawing the cells: (i) the cells are reconstituted or dilated before administering the cells to a recipient, or (ii) the cells are directly infused into recipient.

Also embraced is the above method, wherein the plasma component of the whole placental neonatal blood composition is defined as 100%, and wherein the sum of the plasma component of the stored cells of: (i) the white blood cell-enriched fraction and (ii) the RBC-enriched fraction, and (iii) the plasma fraction, is at least 90% or at least 95%. In another aspect, the sum is at least 70%, at least 75%, at least 80%, at least 85%, and the like.

Also provided is the above method, wherein the plasma component of the whole placental neonatal blood composition is defined as 100%, and wherein the sum of the plasma component of the stored cells of: (i) the white blood cell-enriched fraction and (ii) the RBC-enriched fraction, and (iii) the plasma fraction, is lower than 80%, lower than 50%, lower than 20%, or lower than 10%. In other embodiments, the percentage can be lower than 90%, lower than 75%, lower than 70%, lower than 65%, lower than 60%, lower than 55%, lower than 45%, lower than 40%, lower than 35%, lower than 30%, and the like.

Furthermore, what is provided is the above method, further comprising administering the cells from the white blood cell-rich fraction to a subject, and administering the cells from the RBC-rich fraction to the same subject. Moreover, what is provided is the above method, wherein the cells from the white blood cell-rich fraction are administered before administering before the cells from the RBC-rich fraction, or wherein the cells from the RBC-rich fraction are administered before administering the cells from the white blood cell-rich fraction.

In composition embodiments, what is provided is a composition comprising a white blood cell-rich fraction prepared by the above method. What is provided also, is a composition comprising a RBC-rich fraction prepared by the above method. Also provided is a composition comprising a white blood cell-rich fraction and a RBC-rich fraction, both prepared by the above method. Also provided is a composition comprising a plasma fraction prepared by the above method.

In system embodiments, what is provided is a system comprising one or more of the above compositions. In other system embodiments, what is provided is the above system, where one or more of the compositions has been frozen. In yet other system embodiments, what is provided is the above system, wherein one or more of the compositions has been frozen but never thawed.

Regarding numbers of cells, a number can be enumerated as the number of cells that is the sum of living cells, lysed cells, and cell ghosts. Alternatively, the number of cells can be enumerated as the number of living cells, but excluding lysed cells and excluding cell ghosts. Either of these definitions can be used for calculating recovery, where the choice can be based on the context.

The above method, wherein the whole placental neonatal blood composition comprises one or more cryoprotectants for the cryopreservation of the white blood cell-enriched fraction and the red blood cell-enriched fraction, and wherein the one or more anticoagulants is one or more of dimethylsulfoxide (DMSO) plus or minus Dextran sulfate, or glycerin, whereas any plasma fractions can be cryopreserved without any cryoprotectants.

The above method, wherein the whole placental neonatal blood composition comprises one or more cryoprotectants for the cryopreservation of the white blood cell-enriched fraction and the red blood cell-enriched fraction, and wherein the one or more anticoagulants is one or more of dimethylsulfoxide (DMSO) plus or minus Dextran sulfate, or glycerin, whereas any plasma fractions are cryopreserved using either a controlled rate freezing device or a dump freeze method that slowly lowers the temperature from ambient or +4 C to −40 C or −50 C (past the transition phase whereby the DMSO changes from liquid to solid phase), usually around −1 C or −2 C per minute; thereafter, from around −40 C or −50 C to around −90 C to −193 C, the temperature lowering can be as fast as around −100 per minute.

The advantages of the present disclosure to reduce space and volume during storage, to reduce the amount of DMSO, therefore reducing adverse events after administering. Advantages include greater cell recovery as compared, for example, to an embodiment where whole blood is fractionated into several roughly equal fractions, and then processed. Another advantage is where washing is only on red blood-enriched fraction, which reduces loss of white blood cells. Another advantage as compared with plasma reduction/depletion method is to reduce the amount of DMSO, reduce the amount of cell debris, reduce cytokine release, and thereby to reduce adverse events. Another advantage as compared to red blood cell reduction method, is to get better engraftment rate, better overall survival and better disease-free survival. Another advantage of the present disclosure is to reduce mortality of patients by reducing the combination of DMSO, cell debris, and cytokines.

### BRIEF DESCRIPTIONS OF THE FIGURES

FIG. 1 discloses procedure and blood cell compositions where a density gradient is used.

FIG. 2 shows procedure and blood cell compositions, where a cell fractionator is used.

FIG. 3. The flow chart discloses procedures where RBC aggregant is added. FIG. 3A involves an upright blood bag and FIG. 3B involves an inverted blood bag.

As used herein, including the appended claims, the singular forms of words such as “a,” “an,” and “the” include their corresponding plural references unless the context clearly dictates otherwise. All references cited herein are incorporated by reference to the same extent as if each individual publication, patent, and published patent application, as well as figures and drawings in said publications and patent documents, was specifically and individually indicated to be incorporated by reference.

### DEFINITIONS

“Administration” as it applies to a human, mammal, mammalian subject, animal, veterinary subject, placebo subject, research subject, experimental subject, cell, tissue, organ, or biological fluid, refers without limitation to contact of a blood cell composition, an exogenous ligand, reagent, placebo, small molecule, pharmaceutical agent, therapeutic agent, diagnostic agent, or composition to the subject, cell, tissue, organ, or biological fluid, and the like. “Administration” can refer, e.g., to therapeutic, pharmacokinetic, diagnostic, research, placebo, and experimental methods. Treatment of a cell encompasses contact of a reagent to the cell, as well as contact of a reagent to a fluid, where the fluid is in contact with the cell. “Administration” also encompasses in vitro and ex vivo treatments, e.g., of a cell, by a reagent, diagnostic, binding composition, or by another cell.

Severe Adverse Events (SAEs) and adverse events (AEs) are defined. The FDA's Guidance for Industry provides the following definition of serious adverse events (SAEs): “Any untoward medical occurrence that at any dose: results in death, is life-threatening, requires inpatient hospitalization or prolongation of existing hospitalization, results in persistent or significant disability/incapacity, or is a congenital anomaly/birth defect.”

Adverse Events (AE) are defined. The FDA's Guidance for Industry provides the following definition of adverse events (AEs): “An AE is any untoward medical occurrence in a patient or clinical investigation subject administered a pharmaceutical product and that does not necessarily have a causal relationship with this treatment. An AE can therefore be any unfavorable and unintended sign (including an abnormal laboratory finding), symptom, or disease temporally associated with the use of a medicinal (investigational) product, whether or not related to the medicinal (investigational) product.” (see, U.S. Department of Health and Human Services. Food and Drug Administration. Guidance for Industry. E6 Good clinical practice:consolidated guidance (April 1996)). These definitions are used by FDA-regulated clinical trials.

An “agonist,” as it relates to a ligand and receptor, comprises a molecule, combination of molecules, a complex, or a combination of reagents, that stimulates the receptor. For example, an agonist of granulocyte-macrophage colony stimulating factor (GM-CSF) can encompass GM-CSF, a derivative of GM-CSF, an antibody that stimulates GM-CSF receptor. An “antagonist,” as it relates to a relationship between a ligand and receptor, comprises a molecule, combination of molecules, or a complex, that inhibits, counteracts, downregulates, and/or desensitizes the receptor. “Antagonist” encompasses any reagent that inhibits a constitutive activity of the receptor. A constitutive activity is one that is manifest in the absence of a ligand/receptor interaction. “Antagonist” also encompasses any reagent that inhibits or prevents a stimulated (or regulated) activity of a receptor. By way of example, an antagonist of GM-CSF receptor includes, without implying any limitation, an antibody that binds to the ligand (GM-CSF) and prevents it from binding to the receptor, or an antibody that binds to the receptor and prevents the ligand from binding to the receptor, or where the antibody locks the receptor in an inactive conformation.

“Effective amount” encompasses, without limitation, an amount that can ameliorate, reverse, mitigate, prevent, or diagnose a symptom or sign of a medical condition or disorder. Unless dictated otherwise, explicitly or by context, an “effective amount” is not limited to a minimal amount sufficient to ameliorate a condition.

“Therapeutically effective amount” is defined as an amount of a reagent or pharmaceutical composition that is sufficient to show a patient benefit, i.e., to cause a decrease, prevention, or amelioration of the symptoms of the condition being treated. When the agent or pharmaceutical composition comprises a diagnostic agent, a “diagnostically effective amount” is defined as an amount that is sufficient to produce a signal, image, or other diagnostic parameter. Effective amounts of the pharmaceutical formulation will vary according to factors such as the degree of susceptibility of the individual, the age, gender, and weight of the individual, and idiosyncratic responses of the individual. See, e.g., U.S. Pat. No. 5,888,530 issued to Netti, et al, which is incorporated herein by reference.

“Extracellular fluid” encompasses, e.g., serum, plasma, blood, interstitial fluid, cerebrospinal fluid, secreted fluids, lymph, bile, sweat, fecal matter, and urine. An “extracellular fluid” can comprise a colloid or a suspension, e.g., whole blood or coagulated blood.

“Growth factor” encompasses factors that stimulate growth, where this encompasses polypeptide and oligopeptide growth factors, polypeptide and oligopeptide hormones, and hormones that are not polypeptides. “Growth factor” also encompasses mutated polypeptide growth factors, or chemically modified small molecule growth factors, that may occur naturally and that have growth factor stimulating ability. Although nutrients such as carbohydrates, fats, minerals, and vitamins are required for growth, these are generally not considered to be growth factors. Polypeptides, peptides, chemicals, small molecules, and compositions that are mimetics of naturally occurring growth factors, but that are not likely to arise naturally, are classified as mimetics.

A composition that is “labeled” is detectable, by spectroscopic, photochemical, biochemical, immunochemical, isotopic, or chemical methods. For example, labels include radioactive isotopes of phosphorous, iodine, sulfur, carbon, stable isotopes, epitope tags, fluorescent dyes, electron-dense reagents, substrates, or enzymes, e.g., as used in enzyme-linked immunoassays, or fluorettes (see, e.g., Rozinov and Nolan (1998) Chem. Biol. 5:713-728). Placental neonatal blood, for example, can be incubated with anti-CD34 antibodies conjugated to fluorescein, or with anti-CD45 antibodies conjugated to phycoerythrin (BD Biosciences, San Jose, Calif.).

“White blood cells” comprises lymphocytes, neutrophils, granulocytes, stem cells, progenitor cells, macrophages, dendritic cells, and others.

### DETAILED DESCRIPTION OF THE DISCLOSURE

### Collecting Placental Neonatal Blood

Following delivery of an infant, and severance of the umbilical cord, the distal end of the cord can be clamped. A needle can be inserted in the cord followed by withdrawal and collection of the blood. One or more additives can be mixed into and dispersed in the placental neonatal blood. For example, the AS-3 solution contains all of the following ingredients: citrate, citric acid, dextrose, sodium phosphate, sodium chloride, and adenine (Mangel et al (2001) J. Perinatol. 21:363-367). Additives can include one or more anticoagulants, such as heparin (van Der Meer et al (1999) Vox Sang. 77:137-142) or citrate. Coagulants also include citrate-phosphate-dextrose (CPD), and citrate-phosphate-dextrose-adenine (CPDA).

### Processing Placental Neonatal Blood—RBC Aggregants, Density Gradients, Antibodies

The present disclosure encompasses, without implying any limitation, one or more of the following methods and devices for processing placental neonatal blood cells. These methods include use of hetastarch, Sepax® (Biosafe, Houston, Tex.), Lymphoprep® (Axis-Shield, Oslo, Norway), Ficoll-Paque® (Stem Cell Technologies, BC, Canada), Prepacyte-CB® (Bio-E, St. Paul, Minn.); AutoExpress® (Thermogenesis, Rancho Cordova, Calif.).

### Hydroxyethyl Starch

Hydroxyethyl starch (HES) is a class of synthetic colloids that are derived from amylopectin. Polymerized units of D-glucose are joined mostly at 1-4 linkages. The degree of branching is approximately one branch (1-6 linkage) for every 20 units of glucose. This degree of branching is abbreviated as 1:20. Hydroxyethyl groups are added to increase solubility and reduce hydrolysis. Hydroxyethyl starch can be classified according to its molecular weight and by molar substitution. Hydroxyethyl starch has been classified as, hetastarch, hexastarch, pentastarch, and tetrastarch, as detailed below. The pharmacokinetics (PK) of hydroxyethyl starch has been described (see, e.g., Jungheinrich et al (2005) Clin. Pharmacokinet. 44:681-699). Hydroxyethyl starch supports aggregation of red blood cells, resulting in their separation from white blood cells (Henkelman et al (2012) Clin. Hemorheol. Microcirc. 52:27-35; Caines et al (1987) Magn. Reson. Med. 5:67-72).

In embodiments, the present disclosure provides hydroxyethyl starch, for example, hetastarch, at a final concentration of 0.5-1.0%, 1.0-1.5%, 1.5-2.0%, 2.0-2.5%, 2.5-3.0%, 3.0-3.5%, 3.5-4.0%, 4.0-4.5%, 4.5-5.0%, 5.0-5.5%, 5.5-6.0%, 6.0-6.5%, 6.5-7.0%, 7.0-7.5%, or 7.5-8.0%, or any combination thereof, for example, 2.0-6.0%.

Preparations with a molecular weight of 670 kDa (0.75), 600 kDa (0.7), and 480 kDa (0.7) are classified as hetastarch. The molar substitution is indicated in parenthesis. Hexastarch can have a molecular weight of 200 kDa and molar substitution of 0.62. Pentastarch can have a molecular weight of 200 kDa or 70 kDa, each with a molar substitution of 0.5. Tetrastarch has a molecular weight of 130 kDa and molar substitution of 0.42 (Boldt (2009) Anesth. Anaolg. 108:1574-1582).

Hydroxyethyl starch (HES) can be added to and mixed in whole placental neonatal blood as follows. This concerns placental neonatal blood supplemented with anticoagulant. First, calculate the volume of the HES solution to add to the blood bag to give a final ratio of [HES]/[placental neonatal blood units] of 1/5 (vol./vol.). The placental neonatal blood and the HES are mixed by inverting the blood bag several times. The blood bag is connected is placed in a centrifuge. Ensure that the blood bag is free of folds or creases. Centrifuge to give a recovery of at least 60% nucleated cells or at least 80% mononuclear cells. These recovery numbers refer to the recovery that is found in the upper fraction (white blood cell-rich fraction), excluding the lower fraction (RBC-rich fraction). (Total recovery, that is, sum of that in the upper fraction and lower fraction is expected to be 100%.) The skilled artisan is able to configure the centrifugation parameters, that is, revolutions per minute (rpm), radius of centrifugation bucket, and gravities (g), to provide a desired recovery of white blood cells in an upper fraction. Remove collection bag from centrifuge, and use a plasma expressor to remove upper fraction. Before using the plasma expressor, it is optional that the collection bag hang for 15-20 min to allow for additional RBC sedimentation, in order to sharpen the interface between white blood cell-rich plasma and the RBCs. Supernatant is expressed into a processing bag (pages 4-6 to 4-10 of Cord Blood Transplantation Study, Cord Blood Bank Standard Operating Procedures, The EMMES Corp., Rockville, Md.).

### Polygeline

Polygeline, which is a polymer of urea and polypeptides derived from gelatin, and which contains a range of molecules of MW 5,000 to 50,000, can be used as a separation medium (see, e.g., Perutelli et al (1999) Vox Sang. 76:237-240; Davies (1987) Dev. Biol. Stand. 67:129-131).

### Density Gradients

Ficoll-Paque and Lymphoprep have been compared, in terms of numbers of progenitor cells, T cells, B cells, and the like (Yeo et al (2009) Regen. Med. 4:689-696). Hetastarch, plasma depletion, PrepaCyte-CB, Sepax, and Ficoll-Paque, have been compared, in terms of recovery of nucleated cells, in terms of recovery of CD34⁺ hematopoietic progenitor cells, and in terms of removing RBCs and hemoglobin (see, e.g., Basford et al (2009) Cell Prolif. 42:751-761; Basford et al (2010 Int. J. Stem Cells. 3:32-45). Recovery is preferably measured before any freezing of the cells.

Cell fractionator can be used with hydroxyethyl starch, without hydroxyethyl starch, with Ficoll or another density gradient medium, and without any density gradient medium. Available cell fractionators include Sepax (Biosafe, Pittsburgh, Pa.) and Optipress (Baxter Healthcare, Round Lake, Ill.).

Ficoll-Paque is a density gradient medium that contains Ficoll PM400 and sodium diatrizoate. Ficoll PM400 is high molecular weight (MW 400,000) polymer of sucrose and epichlorohydrin. The molecules of Ficoll PM400 are highly branched and compactly coiled. Sodium diatrizoate is the sodium salt of 3,5-diacetamido-2,4,6-triiodobenzoic acid. The present disclosure encompasses methods and systems that use Ficoll-Paque, or another density gradient medium, for separating whole placental neonatal blood into a white blood cell-rich fraction and a RBC-rich fraction. With Ficoll-Paque, a result can the following layers: Platelet layer on top, lymphocyte layer, and at the bottom, layer rich in RBCs and granulocytes (GE Healthcare (May 2007) Isolation of Mononuclear Cells (22 pages)).

The present disclosure, in a non-limiting embodiment, can use Ficoll-Paque to provide a buffy coat layer (enriched in lymphocytes, monocytes, and stem cells), and another layer (enriched in RBCs and that contains some stem cells). According to the present disclosure the two layers are stored frozen separately. In a non-limiting embodiment, the two layers are thawed (but not combined), and are separately infused into the same recipient subject. In a preferred embodiment, cells from the buffy coat layer are thawed and infused first, followed by thawing and infusing cells from the other layer (the layer enriched in RBCs). In another embodiment, the order of thawing and infusing is reversed. In general, embodiments include:

(1) First thawing cells of buffy coat layer and then infusing cells of buffy coat layer, then thawing RBC-rich layer and finally infusing RBC-rich layer;
(2) First thawing cells of buffy coat layer, then thawing RBC-rich layer, then infusing cells of buffy coat layer, and finally infusing cells of RBC-rich layer;
(3) First thawing cells of buffy coat layer, then thawing RBC-rich layer, then infusing cells of RBC-rich layer, and finally infusing cells of buffy coat layer;
(4) First thawing RBC-rich layer, then infusing cells of RBC-rich layer, then thawing cells of buffy coat layer and then infusing cells of buffy coat layer;
(5) First thawing RBC-rich layer, then thawing cells of buffy coat layer, then infusing cells of RBC-rich layer, and finally infusing cells of buffy coat layer;
(6) First thawing RBC-rich layer, then thawing cells of buffy coat layer, then infusing cells of buffy coat layer, and finally infusing cells of RBC-rich layer.
Exclusionary embodiments are also provided, that is, what can be excluded is method that excludes one or more of the above procedures.

### Antibodies for Preparing Cell-Enriched Fractions

In some embodiments, method of preparing blood products and blood products prepared by the method use anti-glycophorin A antibody to cause agglutination of RBCs, facilitating removal of the RBCs. In non-limiting embodiments, method of preparing blood products and blood products prepared by the method use anti-CD15 antibody to agglutinate and remove neutrophils, use anti-CD9 antibodies to remove platelets, or use anti-CD41 antibodies to remove platelets (see US 2010/0028851 issued to Collins, which is incorporated herein in its entirety). In exclusionary embodiments, present disclosure excludes methods and blood products prepared by said methods, that use one or more of anti-glycophorin A, anti-CD15, anti-CD9, and anti-CD41 antibodies.

### Processing Placental Neonatal Blood—Cell Fractionators Sepax

Sepax® (Biosafe America, Houston, Tex.) is an automated virtually enclosed device for processing 35-320 mL of placental neonatal blood, bone marrow, or peripheral blood, which provides a final volume of 20-50 mL, in a processing time of about 35 min. The processing of samples that are over 320 mL can be done in multiple bags, divided in equal or unequal portions. The recovery of total nucleated cells is over 85%, and recovery of CD34⁺ cells is over 90%. The device provides three fractions: hematopoietic cell concentration, plasma, and red blood cells. Sepax suggests using hydroxyethyl starch (Product Data Sheet, Product #14200—UCB-HES, Biosafe). Fractionation can be achieved without hydroxyethyl starch. Recovery is preferably measured prior to any freezing of the cells. The Sepax machine contains a centrifugal processing chamber that processes blood by displacement of an axially moveable piston (U.S. Pat. No. 6,123,655 issued to Fell), which is incorporated herein by reference.

Cell fractionator can provide white blood cell-rich fraction and RBC-rich fraction where buffers and solutions do not contain any sedimentation agent, such as a RBC aggregant (page 4C-2 of Biosafe (January 2008) SEPAX Cell Processing System Operator's Manual, 208 pages).

The present disclosure encompasses use of a machine with components that are the same as, that are structurally similar to, and that are functionally similar to, those of Sepax. What is also encompassed is blood cell products prepared by this type of machine, or by similar machine. Sepax includes a centrifugal processing chamber, which functions as a spinning syringe, where its volume is variable by away of a piston. The piston is actuated by vacuum or pressure through a port located at the bottom of the processing chamber. The piston moves freely in the rotor of the processing chamber. An inlet/outlet port is located through the upper axis of the rotor. Blood passes through a rotating seal and enters a separation space, where transfer is helped by centrifugal pumping, where the blood or other fluid that is introduced under rotation exerts a pressure on the piston that increases with centrifugation speed. The centrifuged biological fluid deposits in layers, with the less dense matter moving to the inner region of the separation space. With blood, the layers are RBC-enriched fraction on the outside, intermediate layer enriched in white blood cells, and plasma as inside layer (U.S. Pat. No. 6,123,655 of Fell). The present disclosure encompasses a method for using a machine, and blood cell products prepared by this machine, where the machine comprises a centrifugal processing chamber, a piston, a rotor, and a vacuum/pressure port, and inlet/outlet port for fluid such as blood.

### PrepaCyte

PrepaCyte-CB® is a device composed of three attached processing and storage bags containing PrepaCyte-CB separation solution. The system's interconnected, closed-bag set limits cell manipulation. PrepaCyte-CB is used for recovery of total nucleated cells (TNCs), mononucleated cells (MNCs) and CD34⁺ progenitor stem cells from human umbilical placental neonatal blood, and depletion or removal of red blood cells. PrepaCyte-CB causes unwanted cells to settle to the bottom of a container/bag, leaving desired cells in the upper fraction of the solution.

### AutoXpress

AXP AutoXpress® Platform (Thermogenesis Corp., Rancho Cordova, Calif.). AXP AutoXpress Platform is an automated, virtually closed system that reduces placental neonatal blood volume to 20 mL in less than 40 min, while retaining greater than 97% mononucleated cells (MNCs). The AXP Platform consists of the AXP™ device, docking station, processing set, and software.

A comparison between an in-house red cell production procedure (St. Louis Univ. Cord Blood Bank), and procedures using PrepaCyte-CB, Sepax, and AXP AutoXpress is available (Henderson et al (2010) Evaluation of Processing Technologies for Umbilical Cord Blood (UCB), Abstract. ISCT Meeting: 2010 May, Phila., Pa.).

### Elutriator

The disclosure provides methods and blood cell compositions provided by counterflow centrifugal elutration. Elutration can be used to prepare white blood cell-enriched fractions and RBC-enriched fractions, and to prepare reduced-volume cell fractions (see, e.g., Donaldson et al (1997) J. Immunol. Methods. 203:25-33; Dodek et al (1991) In Vitro Cell Dev. Biol. 27A:211-214; Mason et al (1985) Scand. J. Haematol. 34:5-8; Gengozian et al (1998) Transplantation. 65:939-946; Lasch et al (2000) Clin. Chem. Lab. Med. 38:629-632). Centrifugal elutriation combines centrifugation with counterflow elutriation (separation by washing). Each biological cell in the chamber of the elutriator encounters centrifugal force, driving the cell away from the axis of rotation, and flowing fluid, which drives the cell towards the axis of rotation. Small cells are washed towards the axis, where they are washed out of the chamber to a collection vessel. Larger or denser cells move more slowly, and reach equilibrium position. The largest or densest cells remain near the inlet to the chamber. By increasing flow, successive fractions can be washed out and collected (Beckman Coulter (June 2012) JE-5.0 Elutriation system (80 pages).

### Centrifugation

In embodiments, the disclosure provides centrifugation at, for example, 50 g for 10 min, 100 g for 10 min, 125 g for 10 min, 150 g for 10 min, 175 g for 10 min, 200 g for 10 min, 250 g for 10 min, 300 g for 10 min, 350 g for 10 min, 400 g for 10 min, 450 g for 10 min, 500 g for 10 min, 550 g for 10 min, 600 g for 10 min, 650 g for 10 min, 700 g for 10 min, 750 g for 10 min, 800 g for 10 min, 1000 g for 10 min, 1200 g for 10 min, 1400 g for 10 min, 1500 g for 10 min, 1600 g for 10 min, 1800 g for 10 min, 2000 g for 10 min, 2200 g for 10 min, 2400 g for 10 min, 2500 g for 10 min, and the like. Ranges of these are also provided, that is, ranges that provide an option between centrifugation at 400-500 g for 10 min. In alternate embodiments, centrifugation can be for, e.g., 5 min, 15 min, 20 min, 30 min, 40 min, 60 min, 80 min, 100 min, 120 min, and so on. Ranges of timings are also provided, e.g., centrifugation for 10-15 min, or 10-20 min. The centrifuge brake can be left on. Or the brake can be left off or set on low breaking, to reduce disruption of cell-rich fraction during deceleration. In embodiments, centrifugation is at about 2 degrees C., about 5 degrees, about 10 degrees, 15 degrees, 20 degrees, 23 degrees (room temperature), or at ambient temperature. In some embodiments, blood bag is cooled on ice before placing in the centrifuge, while in other embodiments, blood bag is allowed to cool inside refrigerated centrifuge before initiating centrifugation. Gravity sedimentation is also an option. For any quantity recited herein, the term “about” can be used, where “about” can mean, e.g., +/−5%, +/−10%, +/−15%, +/−20%, +/−25%, +/−50%, and the like.

### Red Blood Cell Ghosts

Red blood cells (RBCs) and RBC ghosts can be characterized and quantitated by, for example, by shape, staining, osmometry, permeability, light scattering, and ion transport (see, e.g., Hoffman (1958) J. Gen. Physiol. 42:9-28; Chang et al (1983) Biochim. Biophys. Acta. 731:346-353; Greer et al (2008) Wintrobe's Clinical Hematology, 12^{th} ed., Lippincott, Williams, and Wilkins, pp. 128-132, 151-152, 984-985, 1032-1041). In embodiments, the present disclosure results in a preparation where the ratio of RBC ghosts to intact RBCs is greater than 0.001, greater than 0.02, greater than 0.05, greater than 0.10, 0.20, 0.50, 1.0, 2.0, 5.0, 10, 20, 50, 100, 200, 500, 1,000, 2000, 5000, 10000, 20000, 50000, 100000, or greater than one million, and the like.

### Hemolysis

In embodiments, what is provided are methods, and blood products produced by these methods, with reduced free hemoglobin. Hemolysis can be increased, for example, by mechanical energy, storage, or freeze/thawing. Free hemoglobin released by hemolysis can be detected by any pink or red discolorization by the naked eye, or by spectrophotometry, e.g., at 562 nm, 578 nm, and 598 nm (Laga et al (2006) Am. J. Clin. Pathol. 126:748-755; Blakney et al (1975) Clin. Biochem. 8:96-102). The present disclosure provides a composition that comprises white blood cells, where the concentration of soluble hemoglobin is less than 700 mg/dL, less than 600 mg/dL, less than 500 mg/dL, less than 400 mg/dL, less than 300 mg/dL, less than 200 mg/dL, less than 100 mg/dL, less than 90 mg/dL, less than 80 mg/dL, less than 70 mg/dL, less than 60 mg/dL, less than 50 mg/dL, less than 40 mg/dL, less than 30 mg/dL, less than 20 mg/dL, less than 15 mg/dL, less than 10 mg/dL, less than 5 mg/dL, less than 2 mg/dL, less than 1 mg/dL, and so on. In one aspect, these measurements refer to a suspension of white blood cells that is brought to a concentration of white blood cells that is about identical to the concentration of the white blood cells in the original whole blood. In exclusionary embodiments, the method and blood product prepared by the method can exclude methods and products that fail to meet any of the above cut-off points. The cut-off points can apply to preparations of white blood cells that have not been washed, that have been washed, that have not been frozen, that have been freeze/thawed, any combination of these, and so on. Free hemoglobin can lead to renal damage (Yoshioka et al (1985) J. Trauma. 25:281-281; Ohshiro et al (1980) Res. Exp. Med. (Berl.) 177:1-12). In the present disclosure, thawing is opitionally followed by washing. In an exclusionary embodiment, the present disclosure includes only cells that have been washed after thawing. In another exclusionary embodiment, the disclosure excludes any cells that have not been washed after thawing.

### Reducing the Amount of Plasma

The amount of plasma can be reduced, preferably before washing. The disclosure provides, in non-limiting embodiments, method and cells prepared by method, wherein placental neonatal blood composition comprises plasma, where the plasma component of the placental neonatal blood composition is defined as 100%. The sum of the plasma component of the stored cells of the white blood cell-enriched fraction plus the plasma component of the stored cells of the RBC-enriched fraction, can be, e.g., lower than 95%, lower than 90%, lower than 85%, lower than 80%, lower than 75%, lower than 70%, lower than 65%, lower than 60%, lower than 55%, lower than 50%, lower than 45%, lower than 40%, lower than 35%, lower than 30%, lower than 25%, lower than 20%, lower than 15%, lower than 10%, or lower than 5%. Freezing plasma fraction is used, in alternative embodiment, for anti-aging, for cosmetic applications, or for other therapeutic applications.

### Recoveries

In embodiments, the present disclosure provides a recovery of greater than 50×10⁷ total nucleated cells (TNCs), greater than 100, 150, 200, 250, 300, 400, 500×10⁷ TNCs, and the like. In embodiments, what is provided is recovery greater than 10% of the TNCs present in placental neonatal blood samples at collection (or prior to processing), greater than 15%, 20%, 25%, 30%, 35%, 40%, 45%, 50%, 55%, 60%, 65%, 70%, 75%, 80%, 85%, 90%, 95%, 98%, of the TNCs present in placental neonatal blood samples at collection (or at a time prior to processing). In embodiments, the present disclosure provides a recovery of greater than 10 million total nucleated cells (TNCs)/kg body weight, greater than 11 million, 12, 13, 14, 15, 16, 17, 18, 19, 20, 21, 22, 23, 24, 25, 26, 27, 28, 29, 30, 31, 32, 33, 34, 35, 36, 37, 38, 39, or 40 million, or more total nucleated cells/kg body weight (see Basford et al (2009) Cell Prolif. 42:751-761). In other non-limiting embodiments, the present disclosure provides a recovery of greater than CD34⁺ cell numbers of 14,000 cells/kg body wt., 15,000 cells; 16,000 cells; 17,000 cells; 18,000 cells; 19,000 cells; 20,000 cells; 22,000 cells; 24,000 cells; 26,000 cells; 28,000 cells; 30,000 cells; 32,000 cells; 34,000 cells; 36,000 cells; 38,000 cells; or 40,000 CD34⁺ cells/kg body wt., and the like. In embodiments, the disclosure provides a recover of greater than 1×10⁵ CD34⁺ cells, greater than 5, 10, 15, 20, 30, 35, 40, 45, 50, 55, 60, 65, 70, 75, 80, 85, 90, 95, 100, 125, 150, 175, 200, 300, 400, 500, 600, 700, 800, 900, or 1,000×10⁵ CD34⁺ cells. These numbers are adjusted for any samples or aliquots that are withdrawn, for example, for archiving, for analysis of blood chemistry or hematology, or for verification of sample identity. Nucleated cells can be measured, e.g., with CellDyn 4000 (Abbott Diagnostics, Abbott Park, Ill.), or Sysmex XE-9100 hematology analyzer (Sysmex, Japan). CD34⁺ cells can be measured by flow cytometry, e.g., with FACS Caliber (BD Biosciences, San Jose, Calif.). Exclusionary embodiments are provided, that is, those which exclude any procedure (or cells prepared by procedure) that correspond to one or more of the above numbers, or that correspond to a range that can be defined by two or more of the above numbers.

Recovery can be expressed in terms of recovery of white blood cells, as measurable in units of total nucleated cells, total CD34⁺ cells, mononuclear cells, or total colony forming units, as measured prior to freezing or after thawing compared to numbers measured after collection and prior to processing, freezing, and thawing. Recovery can also be expressed in terms of the total of the white blood cells that are frozen and then thawed and actually administered into a subject. In this case, “recovery” refers to the sum total of the white blood cells or total nucleated or mononuclear or CD34⁺ or colony-forming unit cells present in the thawed white blood cell-rich fraction, and of the white blood cells or total nucleated or mononuclear or CD34⁺ or colony-forming unit cells, present in the thawed RBC-rich fraction, where recovery is corrected for any cells withdrawn, e.g., for archival or testing purposes. For quantitating the number of white blood cells in a thawed preparation, white blood cells can be counted, e.g., by a light microscope, hematology analyzer (e.g., Coulter LH500 Hematology Analyzer, Beckman Coulter), or by flow cytometry. Preferably, the percent recovery of white blood cells in thawed preparations, and percent of cells originally present in the whole placental neonatal blood that are administered to a subject, are without regard to whether the cells are alive or dead. The percentage of live cells can be measured with various techniques, such as trypan blue dye exclusion or 7-AAD.

For arriving at a calculation of “recovery,” with reference to the number of white blood cells that were originally in the whole placental neonatal blood, the percentage of cells that are infused into the recipient (sum of infused white blood cell-rich preparation and RBC-rich preparation), can also be calculated from the amount of processed material (or processed substance) that is eventually infused. Aside from samples taken from archival or testing purposes, and aside from plasma (essentially lacking in any cells), what is preferred is that 100% of the cells be infused into the subject. In other words, because the only substances that are removed are: (1) Material for archival or testing purposes; and (2) Plasma, it is the case that the present disclosure provides a system and method for infusing 100% of the cells (minus cells that are removed for archival or testing), that is, nearly 100% recovery. Where recovery refers to cell preparations that are to be infused in a subject, the percent recovery refers to the amount of cells in the blood bags immediately before infusion is commenced.

### Washing

The present disclosure provides methods and steps for washing cells. Also provided are cell preparations that are washed, in particular, washed preparations with a reduced concentration of one or more of hemoglobin, red blood cell ghosts, lysed white blood cells (e.g., lysed neutrophils), released cytokines or chemokines, and cell debris from lysed white blood cells, and of cryoprotectant such as DMSO. Hanks' Buffered Salt solution, human serum albumin solution, phosphate buffered saline (PBS), Dulbecco's medium, Roswell Park Memorial Institute (RPMI) medium (GibcoBRL; Sigma-Aldrich), an isotonic solution, a solution containing dextran, and any combination of the above, can be used as components of a washing solution. Washing can be with low-glucose Dulbecco's modified Eagle medium.

Washing of a cell-rich fraction can be accomplished by soaking, swirling, rocking, inverting, vertical rotation, horizontal rotation, vibrating, or any combination of these. Washed cells can be collected, without limitation, by filtering, centrifugation, antibody-based techniques. In an exclusionary embodiment, the method excludes one or more or all washing steps.

Regarding washing, any RBC-rich fraction is preferably washed in order to reduce the concentration of free hemoglobin, RBC ghosts, lysed white blood cells, released cytokines or chemokines, or cell debris from lysed white blood cells, or cryoprotectant. In embodiments, one volume of RBC-rich cells (“original volume”) is mixed with one volume of diluent, then centrifuged, and then re-suspended. In other embodiments one volume RBC-rich cells is mixed with 2 vol., 3 vol., 4 vol., 5 vol., 6 vol., 7 vol., 8 vol., 9 vol., 10 vol., 12 vol., 14 vol., 16 vol., 18 vol., 20 vol., and the like of diluent. Preferably, dilution is with 7 volumes or greater. Following dilution, preparation is centrifuged to produce a cell-rich fraction, and then re-suspended in the original volume using diluent. Washing is preferred where a recipient subject has impaired renal function, or where the recipient has a low body weight, e.g., is a child or infant, or where the recipient has known sensitivity to DMSO or to frozen cell products.

### Administration

For administration of the thawed fractions into a recipient, cells from the white blood cell-rich fraction can be infused first, followed by cells from the RBC-rich fraction (which contains some white blood cells) cam infused second into the same subject. In another embodiment, cells from the RBC-rich fraction are infused first, followed by infusing cells from the white blood cell-rich fraction. In an alternate embodiment, each fraction can be divided into two or more aliquots, where infusion involves a first infusion of an aliquot of cells from the white blood cell-rich fraction, then RBC-rich, then white blood cell-rich, then RBC-rich, and so on. Alternatively, the white blood cell-rich fraction can be infused, and the RBC rich-fraction saved for Donor Lymphocyte Infusion (DLI).

### Timing of Administration

Regarding time of a white blood cell-rich fraction and a RBC-rich fraction, the white blood cell-rich fraction is preferably administered first. Initiation of the white blood cell-fraction and of the RBC-rich fraction can be simultaneous, or can be separated by one minute, two minutes, ten min, 20 min, 30 min, 60 min, 2 hours, 3 h, 4 h, 5 h, about 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 24 h, 2 days, 3 days, 4 days, 5 days, 6 days, 7 days, months, or years, and the like. What is also encompassed are ranges of separation, where any given time is +/−10% of that time, +/−20% of that time, +/−50% of that time, and so on.

In exclusionary embodiments, the present disclosure excludes any preparation (or method preparation) where the recovery is less than: 10 million total nucleated cells (TNCs)/kg body weight, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 million TNCs/kg body weight. In other exclusionary embodiments, what is excluded is any preparation with a recovery less than (or a method having a recovery that is less than): CD34⁺ cell numbers of 14,000 cells/kg body wt., 15,000 cells; 16,000 cells; 17,000 cells; 18,000 cells; 19,000 cells; 20,000 cells; 22,000 cells; 24,000 cells; 26,000 cells; 28,000 cells; 30,000 cells; 32,000 cells; 34,000 cells; 36,000 cells; 38,000 cells; or 40,000 CD34⁺ cells/kg body wt.

Regarding the present disclosure, what is encompassed is recovery of total nucleated cells (TNC) of at least 85%, at least 90%, at least 91%, at least 92%, at least 93%, at least 94%, at least 95%, at least 96%, at least 97%, at least 98%, at least 99%, and so on. What is also encompassed is recovery of CD34⁺ cells of at least 85%, at least 90%, at least 91%, at least 92%, at least 93%, at least 94%, at least 95%, at least 96%, at least 97%, at least 98%, at least 99%, at least 99.5%, and so on. Moreover, what is provided is recovery of colony forming units of at least 85%, at least 90%, at least 91%, at least 92%, at least 93%, at least 94%, at least 95%, at least 96%, at least 97%, at least 98%, at least 99%, at least 99.5%, and so on. Also, what is provided is recovery of white blood cells at least 85%, at least 90%, at least 91%, at least 92%, at least 93%, at least 94%, at least 95%, at least 96%, at least 97%, at least 98%, at least 99%, at least 99.5%, and so on. In exclusionary embodiments, what is excluded is any blood product, or any method that provides a blood product, where recover fails to meet one or more of the above cut-off points. The cut-off points refer to recoveries that are measured prior to freezing. Also, the cut-off points are corrected for by small samples that are withdrawn, e.g., for identifying blood donors, for hematology tests, for clinical chemistry tests, and so on. Thus, the recovery that is first calculated by the laboratory technician is to be subsequently increased by a few percent, by way of multiplication, in order to correct for the withdrawn blood samples.

### Identifying Cells

Techniques and equipment for measuring expression, and for identifying cells, include flow cytometry, histology, gene arrays, and reagents such as antibodies, enzyme-linked antibodies, fluorescent antibodies, polymerase chain reaction (PCR), and the like. Guidance on flow cytometry is available (see, e.g., BD Biosciences, San Jose, Calif. (December 2007) BD FACSAria II User's Guide, part no. 643245, Rev.A (344 pages)). CD34⁺ cells can be determined by flow cytometry (FACSCalibur, BD Biosciences, San Jose, Calif.) with CD34-PE and CD45-FITC (BD Biosciences). Viability can be tested by colony-forming unit assays (MethoCult GFH 444, Stemcell Technologies, Vancouver, BC, Canada). Plasma expressors are available from Fenwall, Inc. (Lake Zurich, Ill.), Baxter (Deerfield, Ill.), and AWEL International (Blain, France). A plasma expressor can use an optical sensor to precisely separate plasma from red blood cells after centrifugation. The expressor automatically clamps the tubing once red blood cells are detected in the line, to prevent settled red blood cells from being transferred with the plasma. Blood bags, tubing, and related hospital supplies are available, for example, Baxter Healthcare (Deerfield, Ill.); GenesisBPS (Hackensack, N.J.).

### Identifying White Blood Cells

Placental neonatal blood, bone marrow, and peripheral blood contain white blood cells. White blood cells acquired from these compartments of the body include CD8⁺ T cells, CD4⁺ T cells, dendritic cells or their precursors, B cells, NK cells, regulatory T cells (Tregs), macrophages, neutrophils, progenitor cells, and stem cells. CD8⁺ T cells directly attack host cells that are infected with viruses or bacteria, or that are cancer cells, and destroy the host cells, thereby reducing the burden of the infection or the cancer. CD4⁺ T cells modulate the activity of CD8⁺ T cells, as well as of other cells, for example by expressing various cytokines. B cells secrete antibodies. The phenotypes of the white blood cells from placental neonatal blood and peripheral blood have been compared, for example, in the case of NK cells (Verneris and Miller (2009) Br. J. Haematol. 147:185-191), CD8⁺ T cells and CD4⁺ T cells (Szabolos et al (2003) Exp. Haematol. 31:708-714). Dendritic cells from placental neonatal blood and from peripheral blood have been compared (see, Charrier et al (2012) Cell Immunol. 276:114-121). Regarding Tregs, Tregs from placental neonatal blood may have a more potent suppressor function than Tregs from adults (Brown et al (2008) Clin. Immunol. 127:286-297; Seggewiss et al (2010) Blood 115:3861-3868). Methods of hematology, for example, blood counts, are available (Greer et al. (eds.) (2008) Wintrobe's Clinical Hematology, 12^{th} ed., Lippincott, Williams, and Wilkins, Phila., Pa.). HLA subtyping may be according to the WHO Nomenclature Committee for Factors of the HLA System (see, Marsh et al (2010) Tissue Antigens. 75:291-455). The following concerns enumeration of cells by “colony forming units” (CFU). Colony growth can be enumerated using a microscope and scored according to CFU-GM (colorless appearance), CFU-GEMM (fried egg appearance), or BFU-E (bright red appearance) (pages 6-11, 6-12 of Cord Blood Transplantation Study, Cord Blood Bank Standard Operating Procedures, The EMMES Corp., Rockville, Md.).

### Cryoprotectant Solutions

A non-limiting, cryoprotectant solution is: 50% DMSO and 5% dextran sulfate (Gentran-40), pre-chilled to 4 degrees, added to a final concentration of 5% to 10% DMSO, and 0.5-1.0% dextran sulfate. Gentran-40 is a dextran with an average molecular weight of 40,000 Daltons (Chow et al (2007) Biology of Blood and Marrow Transplantation. 13:1346-1357; Petz et al (2012) Transfusion. 52:1311-1320; U.S. Pat. No. 8,062,837 issued to Chow, which is incorporated herein in its entirety). The cryoprotectant solution can be added using a syringe pump. Freezing bags, cryovials, filters, membranes, and other supplies are available (Pall Medical Corp., Port Washington, N.Y.).

### Controlled Freezing

After adding cryoprotectant, placental neonatal blood products can be frozen by controlled freezing. A non-limiting, method is freezing from 4 degrees to −50 degrees, dropping one degree per minute, then from −50 degrees C. to −90 degrees C., at a rate dropping ten degrees per minute (Chow et al (2007) Biology of Blood and Marrow Transplantation. 13:1346-1357; Petz et al (2012) Transfusion. 52:1311-1320). Once at −90 degrees C., the placental neonatal blood products are transferred to liquid nitrogen. Freezing program can involve a program of 1 degree C/min to 2 degrees C/min, starting at 4 degrees to −40 degrees, and after that, 10 degrees C. per min down to −90 degrees (page 4-17 of Cord Blood Transplantation Study, Cord Blood Bank Standard Operating Procedures, The EMMES Corp., Rockville, Md.). After reaching −90 degrees, the frozen blood product is then transferred to liquid nitrogen storage, either in liquid phase, vapor phase, or a combination thereof, e.g., first vapor phase then to liquid phase. Freezing can involve starting at 10 degrees C., with cooling at 10 degrees/min until a temperature of minus 3 degrees C. is reached, then cooled at minus 2 degrees/min until minus 50 degrees C. is reached, and then stored under (or stored over) liquid nitrogen (Rodgriguez et al (2004) Vox Sanguinis. 87:165-172).

### Expansion of White Blood Cells

Methods for expansion of white blood cells from placental neonatal blood, bone marrow, and peripheral blood, are available. The term “expansion” refers to cell division to produce a greater number of cells. For example, T cells from placental neonatal blood have been expanded (see, e.g., Skea et al (2004) J. Hematotherapy. 8:129-139). Dendritic cells from placental neonatal blood can be expanded (Harada et al (2011) Sci. Rep. 1:174 (8 pages)). Stem cells from placental neonatal blood can be expanded (see, e.g., Hofmeister et al (2007) Bone Marrow Transplant. 39:11-23).

### Thawing and Administering White Blood Cells

Thawed placental neonatal blood products (plus or minus reconstitution or washing) containing white blood cells can be infused intravenously. Where frozen cells are used, the cells must be thawed. Procedures for thawing are available. For example, samples in liquid nitrogen can be equilibrated in vapor-phase nitrogen for 1-2 h prior to rapid thawing in a 37 degrees C. waterbath (Taang et al (2001) Tansfusion. 41:344-352). Immediately after being thawed, placental neonatal blood products (WBC-rich fraction, RBC-rich fraction, first WBC-rich fraction and then RBC-rich fraction, or first RBC-rich fraction then WBC-rich fraction) can be infused directly. Dilution and infusion method (without a centrifugation and removal of supernatant step) is called, “Reconstitution or Dilution.” Alternatively, thawed placental neonatal blood product (WBC-rich fraction, RBC-rich fraction, or both of these fractions) can be washed, which consists of the following steps. Immediately after thawing, placental neonatal blood products can be diluted with an equal volume of a solution containing 2.5% (wt./vol.) human albumin and 5% (wt./vol.) dextran-40 in isotonic salt solution, with continuous mixing, and then centrifuged at 400×g for 10 min. The 1:1 dilution of the diluted product volume to diluent can be repeated once, or multiple times, preferably twice or more, yielding a final dilution of 1:7 (thawed cell product to diluent) or 8-fold, centrifuged at 400×g for 10 min. The supernatant is removed, and the sedimented cells are resuspended slowly in fresh albumin/dextran solution to a volume appropriate for infusion (Rubinstein et al (1995) Proc. Natl. Acad. Sci. 92:10119-10122). Thawing can be accomplished by warming in a 37 degree C. water bath.

In embodiments, the disclosure provides a post-thawing recovery of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, and the like. Recovery can be measured in terms of colony forming units (CFU), white blood cells, mononuclear cells, total nucleated cells (TNC), CD34⁺ cells, viable cells, and so on. In embodiments, what can be excluded from the compositions and methods of the present disclosure, are compositions and methods that provide a recovery (as measured after thawing) of less than 98%, less than 95%, less than 90%, less than 85%, less than 80%, less than 75%, less than 70%, less than 65%, less than 60%, less than 55%, less than 50%, less than 45%, less than 40%, less than 35%, and the like.

The number of white blood cells that is administered to a subject can be expressed in terms of total nucleated cells/kg body weight, CD34⁺ cells/kg body weight, CD3⁺ cells/kg body weight, CF/kg body weight, or mononuclear cells/kg body weight. Regarding CD34, this protein (CD34) is expressed on a subset of progenitor cells. CD34 expression is used to estimate the number of progenitor cells and stem cells in a blood sample (see, e.g., Grinstein et al (2007) J. Biol. Chem. 282:12439-12449; Mackie et al (2011) Tex. Heart Inst. 38:474-485).

The number of cells that can be administered in total, without limitation, is a number that is about, or alternatively, a number that is greater than, 1 million, 2 million, 5 million, 10 million, 15 million, 20 million total nucleated cells (TNC)/kg body wt., as counted at collection. Counting can be at a time that is pre-processing, post-processing, or post-thawing. What can be administered is greater than 20 million, greater than 25 million, 30 million, 35 million, 40 million, 45 million, 50 million, 55 million, 60 million, 65 million, or greater than 70 million total nucleated cells/kg body wt., and the like. Also, what can be administered is 20-25 million total nucleated cells/kg body wt., 25-30 million, 30-35 million, 35-40 million, 40-45 million, 45-50 million, 50-55 million, 55-60 million, 65-70 million total nucleated cells/kg body wt., and the like.

The present disclosure provides a dose for a recipient of about one million nucleated cells/kg body wt., about 2, about 3, about 4, about 5, about 6, about 7, about 8, about 9, about 10, about 12, about 14, about 16, about 18, about 20, about 22, about 24, about 26, about 28, or about 30, or over 30 million nucleated cells/kg body wt. In other embodiments, what is provided is a dose for a recipient of 2-3 million nucleated cells/kg body wt., 3-4, 4-5, 5-6, 6-7, 7-8, 8-9, 9-10, 10-12, 12-14, 14-16, 16-18, 18-20, 20-22, 22-24, 24-26, 26-28, 28-30 million nucleated cells/kg body wt., and so on.

In embodiments, where a freshly drawn sample of placental neonatal blood (or an equivalent composition of pooled placental neonatal blood) contains X nucleated cells (100%), the present disclosure provides a composition of processed placental neonatal blood cells. A non-limiting embodiment of the present composition of processed placental neonatal blood cells occurs in two compartments. The sum of the nucleated cells in the two compartments is at least 99% X nucleated cells, at least 98% X nucleated cells, at least 97% X nucleated cells, at least 96% X nucleated cells, at least 95% X nucleated cells, at least 90% X nucleated cells, at least 85% X nucleated cells, and the like.

In exclusionary embodiments, what can be excluded is any composition (occurring as one, two, three, or more compartments) that has less than 99% X nucleated cells, less than 98% X nucleated cells, less than 97% X nucleated cells, less than 96% X nucleated cells, less than 95% X nucleated cells, less than 90% X nucleated cells, less than 85% X nucleated cells, and the like. The term “compartments” can refer to white blood cell-rich fraction and RBC-rich fraction. Alternatively, or in addition, “compartments” can refer to blood preparations separately stored in blood bag #1, blood bag #2, blood bag #3, and so on.

### Adverse Events

A number of adverse events have been associated with infusing white blood cells into human subjects. One type of adverse event is the disorder, graft versus host disease (GVHD). Criteria for defining the severity of chronic GVHD are available (see, e.g., Shulman et al (1980) Am. J. Med. 69:204-217; Greinix et al (2011) Biol. Blood Marrow Transplant. 17:167-175). Criteria for assessing the severity of acute GVHD have been published (see, e.g., Przepiorka et al (1995) Bone Marrow Transplant. 15:825-828). Acute GVHD may be characterized by skin rash, jaundice (hepatitis), and abdominal pain and diarrhea, while chronic GVHD often has multi-organ involvement and develops about 100 days after hematopoietic cell transplantation (Flowers et al (1999) Hematol. Oncol. Clin. North Am. 13:1091-1112). GVHD assessment can involve Glucksberg grading, or IBMTR Index (Martin et al (1998) Blood. 92:3479-3481). Other adverse events associated with transplants of white blood cells include transmission of viral infections, e.g., of cytomegalovirus and Epstein-Barr virus. Placental neonatal blood transplants can results in a lower incidence of adverse events that are GVHD or infections, as compared with bone marrow transplants (Rubinstein et al (1998) New Engl. J. Med. 339:1565-1577).

With infusion, DMSO can result in adverse events of blood pressure instability, fever, chills, and nausea. DMSO can induce lysis of red blood cells, resulting in nephrotoxicity caused by free hemoglobin ((page F-4 of Cord Blood Transplantation Study, Cord Blood Bank Standard Operating Procedures (April 2003) The EMMES Corp., Rockville, Md.; Sauer-Heilborn et al (2004) Transfusion. 12:907-916). The present disclosure reduces the frequency of these adverse events, that is, among a population of subjects, and reduces the severity of these adverse events. Excessive amounts of DMSO can be defined as 1 gram/kg and greater than 1 gram/kg of recipient's body weight, infused over a short period. These amounts can lead to severe adverse events (SAEs), e.g., hemodynamic instability and death. DMSO and/or thawing can induce lysis of red blood cells.

Serious adverse events (SAEs) associated with placental neonatal blood transplants also includes those arising from dimethylsulfoxide (DMSO) (cryopreservative), red blood cell ghosts, lysed white blood cells, cytokines, and use of high infusion volumes. Depending on the size of the recipient, high infusion volumes may be those that are 75 mL or greater. The present disclosure reduces the rate of one or more of these SAEs, as compared to transplant with control preparation of placental neonatal blood white blood cells.

Donor Lymphocyte Infusion (DLI) and Pigtail Sample from Cells Reserved for DLI

Donor Lymphocyte Infusion (DLI) involves acquiring white blood cells from a donor, administered a first aliquot of the white blood cells to a recipient, storing a second aliquot of the white blood cells, for example, stored cryogenically, until a future need arises. When need arises, thawed cells are administered to the same recipient. For example, when a white blood cell infusion is used to treat cancer, it may be the case that the treatment was largely successful initially, but the original cancer relapsed later. Another indication is where an anti-cancer drug initiates a new cancer (page 324 of Brody T. (2012) Clinical Trials: study design; endpoints & biomarkers; drug safety; FDA & ICH guidelines. Elsevier, Inc., NY, NY).

DLI can be used if there is any residual disease after a transplant, if there are signs of relapse of the disease, or if a new cancer occurs. Responses to DLI have been seen in patients with leukemia, lymphoma and myeloma (see, e.g., Leukaemia and Lymphoma Research (2012) Donor Lymphocyte Infusion. London, UK (16 pages). The present disclosure provides the step of reserving a step for DLI, using white blood cell contents of the second fraction or compartment, and compositions of cells that are stored for DLI. DLI can be used for reduced intensity stem cell transplantation. Use of placental neonatal blood products for DLI has not been described previously. Also provided, is a pigtail that exists (or that is utilized) only on the bag saved for DLI, which is often the bag containing the RBC-enriched fraction. What is provided without limitation is the following, that is, only the DLI bag has a pigtail but the bag containing cells used for immediate transplantation does not necessarily have a pigtail. Pigtails on blood bags are described in U.S. Pat. No. 4,994,039 of Mattson and U.S. Pat. No. 4,369,779 of Spencer, which are incorporated herein in their entirety. In some embodiments, a pigtail has one, two, three, four, or more segments. Advantage of having the pigtails obtained from the bag containing the RBC-enriched fraction is saving additional white blood cell containing stem cells (or progenitor cells) by not using the pigtails from the bag containing the white blood cell-rich fraction. As the RBC-rich fraction is generally discarded in RBC reduction processing, using pigtails obtained from this fraction further maximizes the total recovery of white blood cells, stem cells, and progenitor cells, in addition to the savings facilitated by cryopreserving and infusing the previously discarded RBC-rich fraction.

### DETAILED DESCRIPTION OF FIGURES

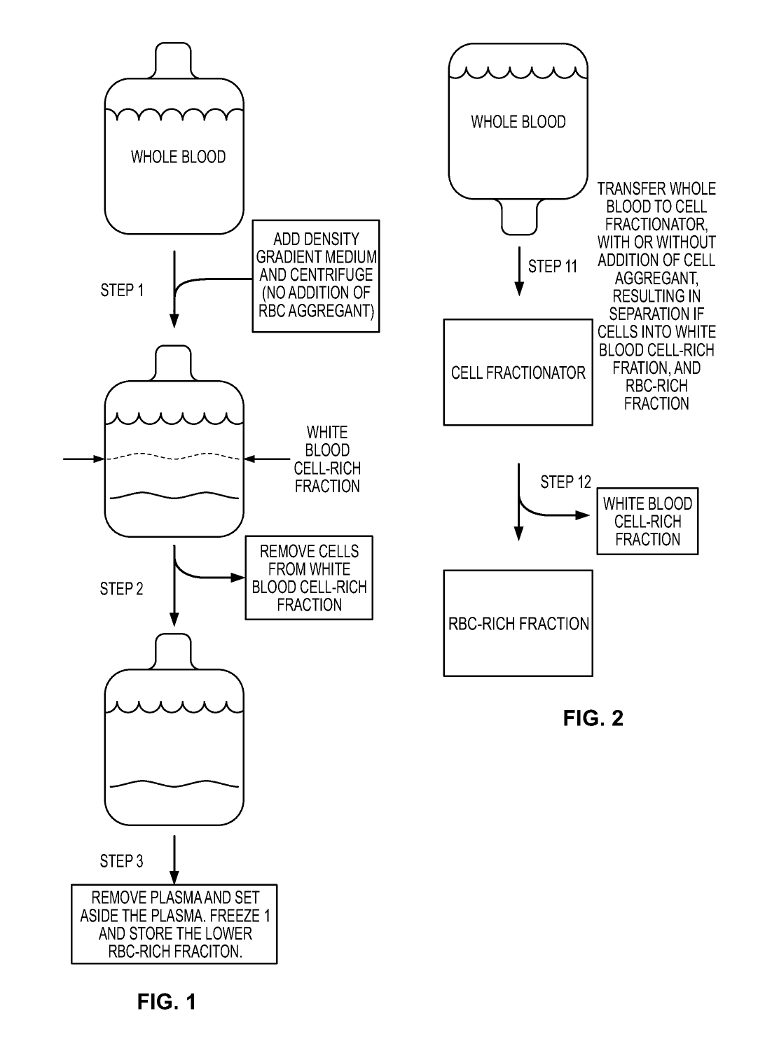

FIG. 1 provides a flow chart that discloses non-limiting methods, and non-limiting blood cell compositions. FIG. 1 involves an upright blood bag. In an alternative embodiment, an inverted blood bag can be used. Method and cell fractions prepared by the method are not necessarily limited to methods that use blood bags. In FIG. 1, Step 1 begins with collected whole blood, supplemented with anticoagulant, in a blood bag. The whole blood is processed using a density gradient medium. The blood bag is centrifuged to produce a supernatant (plasma), white blood cell-rich buffy coat layer, and RBC-rich fraction. The density gradient medium can be Ficoll-Paque®, and where Ficoll-Paque is used, the white blood cell-rich layer includes lymphocytes, monocytes, progenitor cells, and stem cells. The RBC-rich fraction can contain a relatively small percentage of the total lymphocytes and a relatively small percentage of progenitor cells and stem cells.

The following steps can include (1) Removal of supernatant fraction, consisting mainly of plasma; (2) Removal of central white blood cell-rich fraction; and (3) Removal of lower RBC-rich fraction. Step 2 shows removal of the white blood cell-rich buffy coat fraction, which will be cryopreserved with the addition of one or more cryoprotectants and stored in cryogenic Dewar. These fractions can be removed using a plasma expressor, using a pipette, by way of draining, or by other methods known to the skilled artisan. Step 3 shows removal of plasma with setting aside of the plasma. Freeze by addition of one or more cryoprotectantss and store the lower RBC-rich fraction. Without implying any limitation, removed plasma can be stored for later infusion into the same subject, that is, the subject receiving the white blood cell-rich fraction. Removed plasma that has been tested to be free of infectious disease and microorganisms can also be used for general medical purposes, such as anti-aging, anti-wrinkle, skin rejuvenation, and the like. This disclosure is a non-limiting embodiment.

FIG. 2 involves a cell fractionator, such as a Sepax® machine, or an elutriator, and the like. Step 11 begins with placental neonatal blood, supplemented with anticoagulant, in a blood bag or other container. The whole placental neonatal blood is processed in the cell fractionator, e.g., Sepax machine, elutriator, and the like. Step 12 discloses the generation of a white blood cell-rich fraction, and an RBC-rich fraction. The RBC-rich fraction contains some white blood cells, but only a small percentage of the white blood cells originally present in the whole blood. In subsequent steps, both fractions can be frozen, stored, and then thawed. This disclosure is also a non-limiting embodiment. Portions of the removed plasma are usually used for certain clinical lab tests, e.g., infectious disease screening or sterility testing. Plasma fraction can be cryopreserved, stored, then thawed for later use.

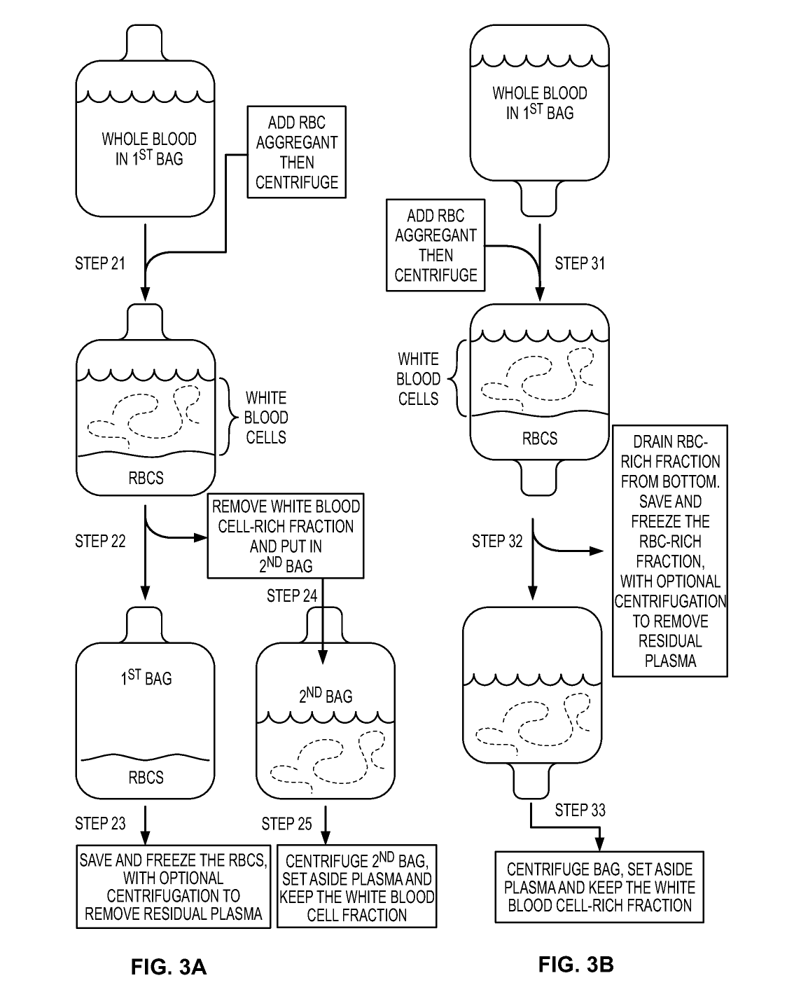

FIG. 3 discloses procedures where a RBC-aggregant is added. FIG. 3A involves an upright blood bag and FIG. 3B involves an inverted blood bag. In FIG. 3A, Step 21 begins with whole blood, supplemented with anticoagulant, in a blood bag (the 1^{st} blood bag). RBC-aggregant is added. The blood bag is centrifuged in this step, providing a supernatant that is rich in white blood cells, and that contains most of the plasma. Centrifugation also results in an RBC-rich fraction, which also contains some white blood cells. Step 22 shows removal of the supernatant, preferably with a plasma extractor, where the supernatant is placed in a 2^{nd} blood bag. Step 23 shows storage of the RBC-rich fraction from the 1^{st} blood bag after transfer into suitable freezing bag and addition of one or more cryoprotectants. Step 24 shows centrifugation of the 2^{nd} blood bag, followed in Step 25 by setting aside the plasma supernatant for testing and storage (without cryoprotectants) and keeping the white blood cell-rich fraction. Regarding the white blood cell-rich fraction from Step 25, the white blood cells are re-suspended and then frozen by the addition of one or more cryoprotectants, subject to cryopreservation and long term storage. This disclosure is another non-limiting embodiment.

For administration, white blood cell-rich fraction can be thawed, and RBC-rich fraction can be thawed, followed by first administering the thawed white blood cell-rich fraction, where next is administering the thawed RBC-rich fraction. The reverse order of administration can also be used. Both fractions a can be simultaneously administered. Delay in administration of the second fraction can be minutes, hours, days, months, or years.

In FIG. 3B, Step 31 begins with whole blood, supplemented with anticoagulant, in a blood bag (the 1^{st} blood bag). RBC-aggregant is added. The blood bag is centrifuged in this step, providing a supernatant that is rich in white blood cells, and that contains most of the plasma. Centrifugation also results in a RBC-rich fraction. The RBC-rich fraction contains some white blood cells. Step 32 shows draining of the RBCs. These RBCs, which contain some white blood cells, are stored in a 2^{nd} blood bag and are frozen. Step 33 starts the 1^{st} blood bag which, at this stage, contains most of the white blood cells. This blood bag is centrifuged in Step 33 to produce a white blood cells-rich fraction, and the plasma supernatant is set aside. The white blood cells are re-suspended and then frozen.

For administration of the compositions of FIG. 3B, white blood cell-rich fraction can be thawed, and RBC-rich fraction can be thawed, followed by first administering the thawed white blood cell-rich fraction, where next is administering the thawed RBC-rich fraction. The reverse order of administration can also be used.

Enriching for White Blood Cells

Techniques available for volume reduction, processing placental neonatal blood include Plasma Depletion/Reduction (PDR) and various methods of Red Blood Cell (RBC) reduction, as outlined below. Although the PDR method does not lead to strict “depletion” of plasma, the term “depletion” is retained here to maintain continuity with past terminology.

### Plasma Depletion/Reduction (PDR)

Plasma Depletion/Reduction (PDR) can involve the following steps (Chow et al (2011) Cytotherapy 1-15; Chow et al (2007) Biology of Blood and Marrow Transplantation. 13:1346-1357):

(1) Collect placental neonatal placental neonatal blood in a collection bag.

(2) Centrifuge the placental neonatal blood in original collection bag. Centrifuge 1680 g for ten minutes at room temperature.

(3) Express the plasma into one of the attached empty bags. This removes plasma from the cells. At this point, the preparation is the plasma depleted/reduced (PDR) placental neonatal blood product.

(4) The collection bag containing the cells is then connected to a freezing bag, and the “plasma depleted/reduced (PDR) placental neonatal blood” is then cooled to 4 degrees, and then one or more cryoprotectants are added.

(5) Freeze to minus 50 degrees, at a slow rate of minus one degree C. per minute.

(6) When cells are needed, thaw, dilute, centrifuge, and remove the supernatant (see, e.g., Rubinstein et al (1995) Proc. Natl. Acad. Sci. 92:10199).

### General Properties Regarding Plasma Depletion/Reduction (PDR) Technique

Placental neonatal blood products cells prepared by the PDR technique used in double cord blood transplantation have been associated with serious adverse events (SAEs), associated with dimethylsulfoxide (DMSO), red blood cell ghosts, lysed white blood cells, cytokines, and very high volumes of infusion, for example, about 150 cc. Adverse events (“cytokine storm”) involving cytokine-induced toxicity have been documented after administering placental neonatal blood transplants (see, e.g., Frangoul et al (2009) Biol. Blood Marrow Transplant. 15:1485-1488). PDR preparations may also present difficulties in thawing of frozen cells. White blood cells prepared by this technique are associated with absolute neutrophil count (ANC) engraftment rates of about 85-90% (Chow et al (2007) BBMT). For the present disclosure, a preferred final concentration of HES is 1.0-1.2% HES.

Regarding the methods and blood products of the present disclosure, infusion of the white blood cells preparations preferably involves, at most, an administration of DMSO at a level of 0.5 grams DMSO per kg body weight of the recipient. An upper level of administratable DMSO is 1.0 g DMSO/kg body wt. of the recipient (Chow et al (2007) BBMT; Chow et al (2011) Transfusion).

### Red Blood Cell (RBC) Depletion/Reduction

### First Example

Red Blood Cell (RBC) reduction can involve the following steps (Rubinstein et al (1995) Proc. Natl. Acad. Sci. 92:10119-10122; Dazey et al (2005) Stem Cells and Development. 14:6-10; Lapierre et al (2007) Cytotherapy. 9:165-169).

(1) Collect placental neonatal blood in a collection bag.

(2) Add hydroxyethyl starch (HES) to a final concentration 1.2%. The HES enhances the sedimentation of the red blood cells.

(3) Centrifuge in original collection bag. Centrifuge at 50 g for five minutes at ten degrees C., in order to acquire a leukocyte-rich supernatant.

(4) After acquiring the leukocyte-rich supernatant, transfer the leucocyte-rich supernatant into a “plasma transfer bag.”

(5) Once the leukocyte-rich supernatant is in the “plasma transfer bag,” centrifuge the “plasma transfer bag” at 400 g for ten minutes in order to sediment and collect the leukocytes. The result is a plasma supernatant and a white blood cell-rich fraction.

(6) Transfer the supernatant (plasma) to a second “plasma transfer bag.”

(7) Finally, re-suspend the leukocytes. Recoveries of cells at each step can be expressed in terms of total number of white blood cells cells, total number of nucleated cells, total number of mononuclear cells, total number of colony forming units, or number of cells expressing a particular biomarker, compared to the number at collection or prior to processing.

### Red Blood Cell (RBC) Depletion/Reduction

### Second Example

Red Blood Cell (RBC) reduction can involve the following steps (Alonso et al (2001) Cryoprotection. 3:429-433).

(1) Collect placental neonatal blood in a collection bag, where collection bag includes anti-coagulant.

(2) Add one volume of hetastarch to 5 volumes of the placental neonatal blood/anti-coagulant mixture.

(3) Bring mixture to 4 degrees C. by placing in a refrigerated centrifuge (without centrifugation) for 45 minutes. Then, centrifuge 5 minutes at 50 g, in order to sediment the red blood cells, and then drain out the red blood cells. This removes about 80% of the red blood cells.

(4) To the supernatant that was above the red blood cells, centrifuge for 13 minutes at 420 g.

(5) Extract the plasma from the top, using a “plasma expressor.” What remains is a product that is depleted in plasma and depleted in red blood cells.

(6) To the product, add enough cold DMSO to give a final concentration of 5-10% DMSO.

### Red Blood Cell (RBC) Depletion/Reduction

### Third Example

Red Blood Cell (RBC) reduction can involve the following steps (Regidor et al (1999) Exp. Hematol. 27:380-385).

(1) Dilute collected placental neonatal blood to 25% hematocrit with Hanks' basic salt solution.

(2) Add 6% (wt./vol.) of HES (molecular weight 450,000) in 0.9% NaCl. The HES is added to 1:7 (vol./vol.) to the blood, for a final HES concentration of 0.75%.

(3) Allow gravity sedimentation of RBC at 22 degrees C. in the first bag, where sedimentation is permitted until a clear demarcation is seen between RBCs and leucocyte-rich plasma.

(4) After the clear demarcation is visible, drain RBCs into a second bag.

(5) Regarding the bag containing the leukocyte-rich plasma, centrifuge this bag at 800 g for ten minutes at 22 degrees C.

(6) After centrifugation, remove the supernatant plasma with a “plasma extractor” to a third bag.

### Red blood Cell Depletion/Reduction

### Fourth Example

The following example is from Basford et al (2009) Cell Proliferation. 42:751-761.

(1) Collect placental neonatal blood and add hetastarch (6% solution) to a volume that is equivalent to 20% of the volume of the placental neonatal blood.

(2) After adding the hetastarch, centrifuge at 125 g for ten minutes.

(3) After centrifugation, remove the supernatant (contains nucleated cells) and place in a second bag, using a plasma expressor, and set aside the RBC-rich fraction.

(4) To the nucleated cells that are in the second bag, centrifuge for 400-500 g for ten minutes, and keep the settled cells (nucleated cells) and remove the supernatant with a plasma expressor and set aside the supernatant.

General properties of Red Blood Cell Depletion/Reduction technique

White blood cells prepared by the Red Blood Cell Reduction technique are characterized by low recovery, for example, recovery where from 15% to 50% of the white blood cells are lost. White blood cells prepared by this technique are associated with lower absolute neutrophil count (ANC) engraftment, for example, where ANC engraftment is only 70-85%.

Current disclosure results in lower AEs and lower SAEs than plasma depletion/reduction, because methods of thawing red cell reduction products can be employed for products manufactured for the current disclosure, and reduce the time required for post-thaw washing or reconstitution/dilution, and hence reduce the amount of cell lysis, and reduce the number of cell lysis products, as compared with PDR placental neonatal blood products. In embodiments, the reduction is to less than 90% of maximal lysis, to less than 80% of maximal lysis, to less than 70% of maximal lysis, to less than 60% of maximal lysis to less than 50% of maximal lysis, to less than 40% of maximal lysis, to less than 30% of maximal lysis, and so on.

Current disclosure, in contrast to red cell reduction products, reduced the amount of cell loss associated with having to wash the entire red cell reduction product after thawing, resulting in significant cell loss. For the current disclosure, an option is to wash just the RBC-rich product.

In embodiments, a method of administration of a composition of the present disclosure, where the first and second preparations of white blood cells are not combined before administration, but are instead administered sequentially and separately, results in lower adverse events. The lowered adverse events can be compared with those occurring where the first and second preparations are combined before administration and are administered to a recipient in this combined form. Also, the lowered adverse events can be compared with those occurring where the white blood cells were prepared using Plasma Depletion/Reduction. In another approach, the lowered adverse events can be compared with those presenting where the white blood cells were prepared by Red Blood Cell (RBC) reduction, or by any other procedure that provides white blood cells from placental neonatal blood, and where the white blood cells are suitable for transplantation into recipient. The lowering of adverse events can be in terms of frequency or in terms of severity. The test group and the comparator group can be chosen to conform to specific demographics, for example, where both test group and comparator group are female (ages 40-60), or where both test group and comparator group were treated by clinics in California. The lowering of adverse events by the composition of the present disclosure, can be 95% or lower than that found with comparator group (in terms of severity, frequency, or any combination), 90% or lower, 85% lower, 80% lower, 75% or lower, 70% or lower, 65% or lower, 60% or lower 50% or lower, and the like.

The control preparation can be from placental neonatal blood prepared by Plasma Depletion/Reduction (PDR) or prepared by Red Blood Cell (RBC) reduction. In embodiments, the present disclosure reduces the amount (in terms of concentration in white blood cell suspension; or in terms of absolute amount administered to a subject) of DMSO, red blood cell ghosts, white blood cell lysis products, one or more cytokines, to 95% or lower, 90% or lower, 85% or lower, 80% or lower, 70% or lower, 60% or lower, 50% or lower, 40% or lower, 30% or lower, 20% or lower, than that using comparator reagents, cells, or methods.

The present disclosure provides reagents, cell preparations, and related methods, for reducing the frequency of AEs in a given population of subjects, for reducing the severity of AEs, and for reducing the number of types of AEs, as compared to reagents, cells, and methods, such relating to Plasma Depletion/Reduction (PDR). Also, the present disclosure provides reagents, cell preparations, and related methods, for reducing the frequency of AEs in a given population of subjects, for reducing the severity of AEs, and for reducing the number of types of AEs, as compared to reagents, cells, and methods, such relating to Red Blood Cell (RBC) reduction. In embodiments, the reduction in frequency or severity is to 95% or lower, 90% or lower, 85% or lower, 80% or lower, 70% or lower, 60% or lower, 50% or lower, 40% or lower, 30% or lower, 20% or lower, than that using comparator reagents, cells, or methods.

The RBC-enriched fraction can be dedicated for donor lymphocyte infusion (DLI). When RBC-enriched fraction is dedicated for DLI, the bloodbag can have a pigtail segment for use in confirmatory typing. Confirmatory typing ensures the identity of the stored sample of blood. Optionally, the DLI can be saved for in vivo expansion for ultimate infusion into the same recipient.

While the method and apparatus have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

It should also be understood that a variety of changes may be made without departing from the essence of the invention. Such changes are also implicitly included in the description. They still fall within the scope of this invention. It should be understood that this disclosure is intended to yield a patent covering numerous aspects of the invention both independently and as an overall system and in both method and apparatus modes.

Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these.

Particularly, it should be understood that as the disclosure relates to elements of the invention, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this invention is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference.

Finally, all references listed in the Information Disclosure Statement or other information statement filed with the application are hereby appended and hereby incorporated by reference; however, as to each of the above, to the extent that such information or statements incorporated by reference might be considered inconsistent with the patenting of this/these invention(s), such statements are expressly not to be considered as made by the applicant.

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC §132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

1. A method for providing fractions from a whole placental neonatal blood composition, wherein the method provides a white blood cell-enriched fraction and a red blood cell (RBC)-enriched fraction, and optionally a plasma fraction,
wherein the RBC-enriched fraction contains more than one white blood cell, wherein the sum of:
(i) the number of white blood cells in the white blood cell-enriched fraction plus
(ii) the number of white blood cells in the RBC-enriched fraction is at least 90% of the total number of white blood cells in the whole placental neonatal blood composition, and
wherein the number of white blood cells in the sum is corrected for any samples that are withdrawn for archival or testing purposes, the method comprising:
(a) processing the whole placental neonatal blood composition to provide a white blood cell-enriched fraction and a RBC-enriched fraction, using a device that is capable of separating cells into the white blood cell-enriched fraction and the RBC-enriched fraction, and optionally the plasma fraction,
(b) cryogenically storing the cells of the white blood cell-enriched fraction and cryogenically storing the cells of the RBC-enriched fraction, and optionally the plasma fraction,
(c) wherein the stored cells of the white blood cell-enriched fraction and the stored cells of the RBC-enriched fraction are capable of administration to one recipient, and wherein said administration is capable of transferring into the recipient at least 90% of the white blood cells, that were derived from the whole placental neonatal blood composition.

2. The method of claim 1, wherein the device comprises a centrifuge or a cell fractionator.

3. The method of claim 1, wherein separation is effected by contacting the whole placental neonatal blood composition with a chemical composition that is capable of separating the whole placental neonatal blood composition into a white blood cell-rich fraction and a RBC-rich fraction, and optionally the plasma fraction.

4. The method of claim 1, wherein separation is effected by contacting the whole placental neonatal blood composition with a chemical composition that is capable of separating the whole placental neonatal blood composition into a white blood cell-rich fraction and a RBC-rich fraction, and optionally the plasma fraction, and
wherein the chemical composition comprises hydroxyethyl starch, density gradient medium, or an antibody.

5. The method of claim 1, wherein the whole placental neonatal blood composition comprises an anti-coagulant.

6. The method of claim 1, wherein the archival or testing purposes comprises one or more of a hematology test, a blood chemistry test, and a donor identification test.

7. The method of claim 1, wherein cells of the white blood cell-rich fraction are administered to the recipient, followed by cells of the RBC-rich fraction being separately administered to the same recipient.

8. The method of claim 1, wherein cells of the RBC-rich fraction are administered to the recipient, followed by the white blood cells of the white blood cell-rich fraction being separately administered to the same subject.

9. The method of claim 1, wherein the whole placental neonatal blood composition comprises one or more anticoagulants.

10. The method of claim 1, wherein the whole placental neonatal blood composition comprises one or more anticoagulants, and wherein the one or more anticoagulants is one or more of citrate and heparin.

11. The method of claim 1, wherein the cells of the white blood cell-enriched fraction are processed by washing to reduce concentration of free hemoglobin, wherein the washing occurs after thawing the cells of the white blood cell-enriched fraction and before administering the cells of the white blood cell-enriched fraction to a recipient.

12. The method of claim 1, wherein the cells of the RBC-enriched fraction are processed by washing to reduce concentration of free hemoglobin, wherein the washing occurs after thawing the cells of the RBC-enriched fraction and before administering the cells of the RBC-enriched fraction to a recipient.

13. The method of claim 1, wherein one or both of the cells from the white blood cell-enriched fraction and the RBC-enriched fraction are not washed, wherein after thawing the cells:
(i) the cells are reconstituted or dilated before administering the cells to a recipient, or
(ii) the cells are directly infused into recipient.

14. The method of claim 1, wherein the plasma component of the whole placental neonatal blood composition is defined as 100%, and wherein the sum of the plasma component of the stored cells of:
(i) the white blood cell-enriched fraction and
(ii) the RBC-enriched fraction, and
(iii) the plasma fraction,
is at least 90% or at least 95%.

15. The method of claim 1, wherein the plasma component of the whole placental neonatal blood composition is defined as 100%, and wherein the sum of the plasma component of the stored cells of:
(i) the white blood cell-enriched fraction and
(ii) the RBC-enriched fraction, and
(iii) the plasma fraction,
is lower than 80%, lower than 50%, lower than 20%, or lower than 10%.

16. The method of claim 1, further comprising administering the cells from the white blood cell-rich fraction to a subject, and administering the cells from the RBC-rich fraction to the same subject.

17. The method of claim 16, wherein the cells from the white blood cell-rich fraction are administered before administering before the cells from the RBC-rich fraction, or
wherein the cells from the RBC-rich fraction are administered before administering the cells from the white blood cell-rich fraction.

18. A composition prepared by the method of claim 1, the composition comprising one or more of:
a white blood cell-rich fraction;
an RBC-rich fraction; and
a plasma fraction.

19-21. (canceled)

22. A system comprising the composition of claim 18.

23. The system of claim 22, where one or more of the compositions has been frozen.

24. The system of claim 22, wherein one or more of the compositions has been frozen but never thawed.

25. The method of claim 1, further comprising, saving a sample for donor lymphocyte infusion (DLI).

26. The method of claim 1, further comprising, administering a DLI.

27. The method of claim 1, wherein the whole placental neonatal blood composition comprises one or more cryoprotectants for the cryopreservation of the white blood cell-enriched fraction and the red blood cell-enriched fraction, and wherein the one or more anticoagulants is one or more of dimethylsulfoxide (DMSO) plus or minus Dextran sulfate, or glycerin, whereas any plasma fractions can be cryopreserved without any cryoprotectants.

28. The method of claim 1, wherein the whole placental neonatal blood composition comprises one or more cryoprotectants for the cryopreservation of the white blood cell-enriched fraction and the red blood cell-enriched fraction, and wherein the one or more anticoagulants is one or more of dimethylsulfoxide (DMSO) plus or minus Dextran sulfate, or glycerin, whereas any plasma fractions are cryopreserved using either a controlled rate freezing device or a dump freeze method that slowly lowers the temperature from ambient or +4 C to −40 C or −50 C (past the transition phase whereby the DMSO changes from liquid to solid phase), usually around −1 C or −2 C per minute; thereafter, from around −40 C or −50 C to around −90 C to −193 C, the temperature lowering can be as fast as around −10 C per minute.
